(19) United States
(12) Patent Application Publication
(10) Pub. No.: US 2016/0074609 A1
(43) Pub. Date: Mar. 17, 2016

(54) TRACHEAL TUBES AND METHODS RELATED THERETO

(71) Applicant: Clayne BENSON, Mars, PA (US)

(72) Inventor: Clayne BENSON, Mars, PA (US)

(21) Appl. No.: 14/855,117

(22) Filed: Sep. 15, 2015

Related U.S. Application Data
(60) Provisional application No. 62/050,304, filed on Sep. 15, 2014.

(51) Int. Cl. A61M 16/04; A61M 16/14

(52) U.S. Cl. CPC A61M 16/0463; A61M 16/0402; A61M 16/0434; A61M 16/145

(57) ABSTRACT

Tracheal tubes capable of delivering local anesthetic to a patient during or after intubation, and methods for making and using the same are described herein.

### CROSS REFERENCE

This application claims priority from U.S. Provisional No. 62/050,304, entitled “Tracheal Tubes and Methods Related Thereto,” filed Sep. 15, 2014, the contents of which are incorporated herein by reference in their entirety.

### SUMMARY

Various embodiments of the invention are directed to a tracheal tube including a tube having longitudinally extending grooves on an exterior surface of the tube, each of the longitudinally extending grooves terminating in a closed end and a semipermeable membrane enclosing the longitudinally extending grooves or mircotubules having a plurality of holes fitted within each of the longitudinally extending grooves. In certain embodiments, the tracheal tube may include a proximal well enclosing a proximal end of each of the longitudinally extending grooves, which may contain a medicament such as, for example, an anesthetic agent. In some embodiments, the tracheal tube may include a port positioned to allow access to the proximal well, and in other embodiments, the proximal well may be seal to enclose the medicament. In particular embodiments, the tracheal tube may include a cuff.

In some embodiments, the semipermeable membrane has a molecular weight cut-off of about 100 Da to about 1500 Da. In some embodiments, the semipermeable membrane may have a sieving coefficient of about 0.6 to about 0.7 for the medicament.

In some embodiments, the tracheal tube may include a compressor or pump to introduce air into the proximal well. In certain embodiments, the microtubules of the tracheal tube may include the holes that are sized to cause nebulization of a medicament in the microtubules when the anesthetic is pressurized. In other embodiments, the compressor or pump may include a venturi and baffle configured to cause nebulization of the medicament in the proximal well.

### BRIEF DESCRIPTION OF THE FIGURES

For a better understanding of the disclosure and to show how the same may be carried into effect, reference will now be made to the accompanying drawings. It is stressed that the particulars shown are by way of example only and for purposes of illustrative discussion of the preferred embodiments of the present disclosure only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings:

FIG. 1 shows cross section of a patient intubated with a tracheal tube.

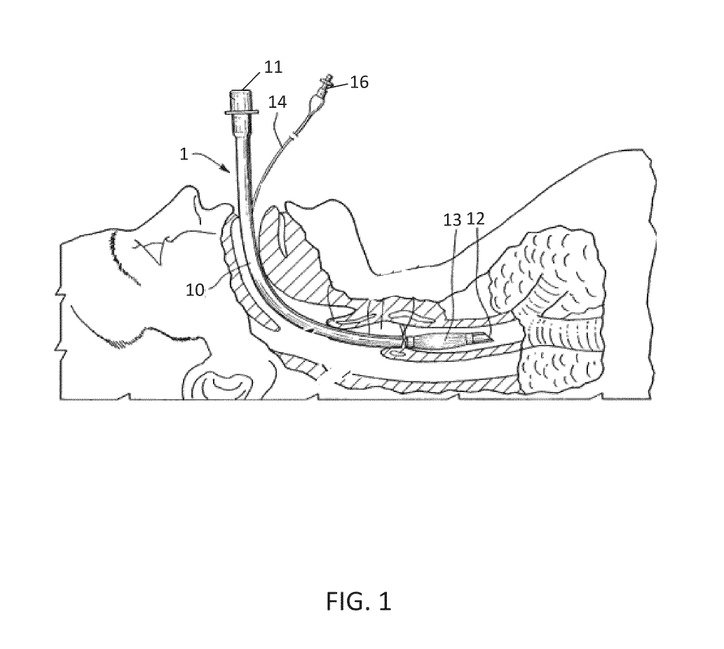

FIG. 2 shows various aspects of the tracheal tube of embodiments of the invention.

### DETAILED DESCRIPTION

Embodiments of the invention are directed to a tracheal tube capable of delivering a local anesthetic agent to a patient during and after insertion. Delirium or acute organic dysfunction of the brain is a common and injurious process encountered in every intensive care unit. The incidence of such dysfunction may increase with age, injury severity, and length of Intensive Care Unit (ICU) stay, and is closely associated with common sedation processes used in ICUs when patients are intubated. Typically, intubated and mechanically ventilated patients are treated with general sedative or hypnotic agents such as versed, Ativan, and propofol to mitigate the anxiety and discomfort associated with intubation. However, sedation is a form of non-restorative sleep in which the patient appears to be deeply sleeping but is unable to enter REM sleep. Over time, the accumulated lack of sleep may lead to delirium. Delivery of local anesthetic agents to tracheal tissue during intubation can minimize or eliminate the need for the use of general sedative agents during mechanical ventilation. The use of such local anesthetic agents may allow patients to remain awake and comfortable during intubation while being able to engage in restorative sleep. In addition to avoiding possible delirium induced by the use of general sedative agents, the use of such local anesthetic agents may also improve the ability of the patient to communicate with attending physicians if necessary.

As used herein, the term “tracheal tube” may include an endotracheal tube, a tracheostomy tube, a double-lumen tube, a bronchoblocking tube, a specialty tube, or any other airway device.

Tracheal tubes, including endotracheal and tracheostomy tubes, are used by physicians for intubation into a patient's trachea to enable the patient to breathe or enable intermittent positive pressure ventilation of the respiratory tract. A typical tracheal tube 1 used in endotracheal intubation may include a curved tube 10 having an open proximal end 11 and an open distal end 12. In some non-limiting examples, the open distal end may be beveled. The tube 10 may be constructed of a flexible material to permit the tube 10 to conform to the shape of the patient's posterior pharynx and trachea as well as to ease the insertion of the tube 10 into the patient. In some non-limiting examples, the tube 10 may have an internal diameter of about 1.0 mm to about 10 mm.

The tracheal tube 1 may include one or more cuffs 13 composed of a thin flexible material surrounding the tube 10 near the distal end 12 of the tube 10. The one or more cuffs 13 can be reversibly inflated from an inflation tube 14 to form a seal between the tube 1 and the wall of the trachea, thereby preventing the escape of air through the trachea away from the lungs and forcing air to flow through the trachea to the lungs. An inflation tube 14 having a proximal port 16 can be disposed outside the tube 10 or within the tube 10 and terminate at the one or more cuffs 13 to provide air for inflating the cuff 14.

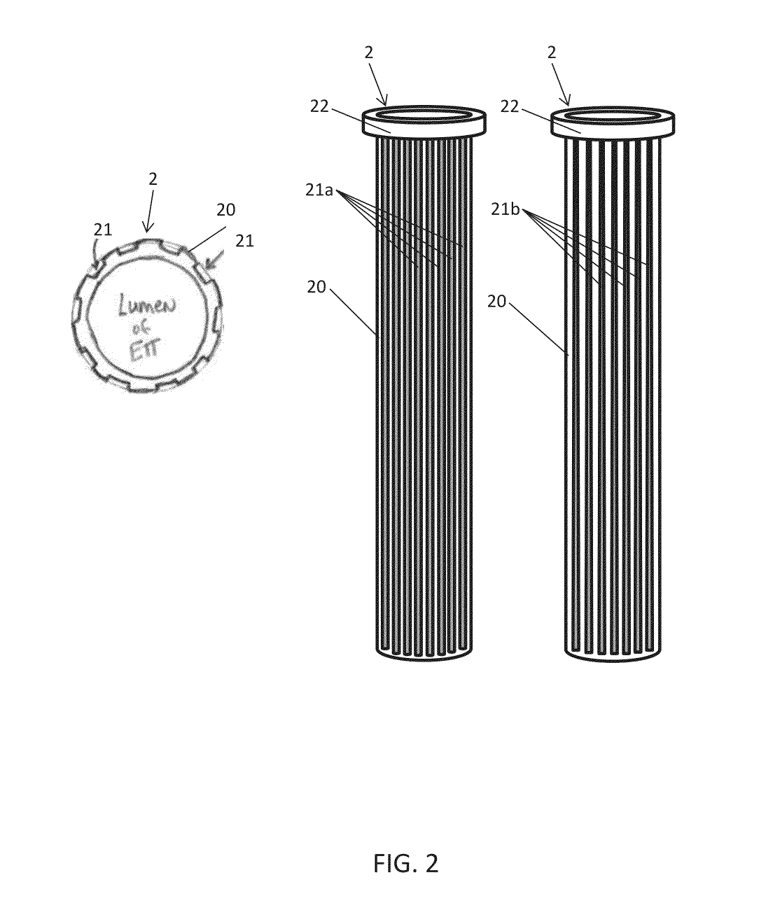

The present disclosure is related to modified tracheal tubes that can deliver anesthetic agents locally during intubation. As illustrated in FIG. 2, various embodiments of the invention may include a tracheal tube 2 having a grooves 21 on an exterior surface of the tube 20. The tube 20 may have any number of grooves 21. For example, in some non-limiting embodiments, the tube 20 may have about 2 grooves 21 to about 25 grooves 21. In other non-limiting embodiments, the tube may have about 4 grooves 21 to about 20 grooves 21 or about 8 grooves 21 to about 15 grooves 21. The grooves 21 may be spaced about the exterior surface of the tube 20 at about equal intervals. For example, a common adult tracheal tube 2 having an outer diameter (OD) of about 10 mm to about 14 mm may have one groove about every 1 mm to about every 1.5 mm around the exterior surface of the tracheal tube. Depending on the OD, the tube 20 may have about 10 grooves 21 to about 22 grooves 21. Based on the thickness of the tube 20, the grooves 21 may have a width of about 0.5 mm to about 1 mm, and a depth of about 0.5 mm to about 1 mm. In a non-limiting example, a typical adult tracheal tube 2 may have a thickness of about 2.5 mm to about 3 mm and can accommodate grooves having widths and depths as disclosed above without loss of physical integrity. However in some alternative non-limiting embodiments, the thickness of the tube 20 may be increased to ensure that the structural integrity of the tube is sufficient to withstand the forces associated with intubation.

In some non-limiting embodiments, the grooves 21 may be uniform grooves 21a having substantially the same width and depth throughout the length of the groove 21a from the proximal end of the tube 20 to the distal end of the tube. In other non-limiting embodiments, the grooves may be tapered grooves 21b that have a narrower width and depth at the distal end of the tube 20b than at the proximal end of the tube 20. In particular embodiments, each groove may include a rounded distal end, and in some embodiments, the distal end of each groove 21 may be squared at the distal end.

The grooves may be enclosed by any means. For example, in some embodiments, microtubules may be pressure fit within the grooves. In other embodiments, the microtubules may be fixed within the grooves by means of an adhesive. The microtubules may have openings throughout their length to allow for release of the contents from the microtubule. In some embodiments, the openings may be dispersed uniformly along the length of the microtubule. In other embodiments, a plurality of openings may be placed at various intervals along the length of the microtubule. For example, a series of about 4 to about 10 openings may be spaced at intervals of about 2 mm to about 5 mm along the length of the microtubule. In some embodiments, regions composed of closely spaced openings on a first microtubule may correspond to regions lacking such openings on a second, neighboring microtubule. In such embodiments, the openings may occur at regular intervals over the entire length of the tube as the tube is rotated.

In certain embodiments, the grooves may be enclosed by a semi-permeable membrane. For example, in some embodiments, a semi-permeable membrane may be attached to the exterior surface of the tube to form a semipermeable sheath so that the grooves may provide an opening through which the local anesthetic agent can travel through the semipermeable sheath. Semi-permeable membranes selectively allow certain molecules to pass through, while others cannot. Movement of molecules across a semi-permeable membrane is determined by concentration gradients, pressure gradients, pore size, molecule size, molecular charge, and the like and combinations thereof. The ability of a substance to pass through a semi-permeable membrane is based on the sieving coefficient for the molecule where a sieving coefficient may range from 0, indicating no transfer of the molecule across the membrane, to 1, indicating unimpeded transfer of the molecule. In various embodiments, the semi-permeable membrane may have a pore size resulting in a sieving coefficient of about 0.6 to about 0.7 for a molecule, thereby allowing passage of some amounts of the molecule across the semi-permeable membrane. In some embodiments, the semi-permeable membranes may have a larger pore size resulting in a sieving coefficients of about 0.9 or about 1.0 for the molecule, thus allowing unimpeded diffusion of the molecule across the membrane.

In some embodiments, a semi-permeable membrane can be characterized by their molecular weight cut-off. A molecular weight cut-off corresponds to a molecule size for which the semi-permeable membrane has a sieving coefficient of 0 or around 0. Typical dialysis membranes may have a molecular weight cut-off of about 100 Daltons (Da) to about 10 million Da. The semi-permeable membranes used in various embodiments can have any molecular weight cut-off. For example, in some embodiments, the molecular weight cut-off may be about 100 Da to about 1500 Da. In certain embodiments, the molecular weight cut-off may be about 200 Da to about 1000 Da or about 300 Da to about 800 Da or any range or individual value encompassed by these ranges, including endpoints.

The semi-permeable membrane may be fabricated from any material known in the art. Such membrane materials include, but are not limited to, celluloses (e.g., regenerated celluloses), cellulose acetates, polytetrafluoroethylenes (e.g., TEFLON™ by DuPont), polysulfones, nitrocelluloses, polycarbonates, polyolefins (e.g., polypropylene, polyethylene, and mixtures thereof), polyamides, polyvinylidene fluorides, and the like. Such membranes may have a pore size of about 0.01 micron to about 1 micron. For example, pore sizes may be about 0.01 microns, about 0.05 microns, about 0.1 microns, about 0.5 microns, or any individual pore size or range encompassed by these ranges including endpoints.

In some embodiments, hollow fibers can be used in place of or in addition to the semi-permeable membrane. In some embodiments, such hollow fibers may be composed of similar materials as the semi-permeable membranes described above and allow molecules of a particular size or less to pass through the fiber walls. Thus, hollow fibers capable of allowing one or more local anesthetic agents to pass through the fibers may be fitted within the grooves in the tubes as disclosed above in relation to microtubule containing tracheal tubes.

The distal end of each groove may be closed so that the groove terminates and the flow of material through the groove must stop at the distal end of the tube. The distal end of each groove may terminate near the distal end of the tube 2, and the proximal end of each groove may be may be enclosed within a proximal well 22 at the proximal end of the tube 2. The proximal well 22 may provide an air and liquid impermeable barrier that can be filled with a local anesthetic agent to be transferred through the grooves. Alternatively, a local anesthetic agent may be transmitted through one or more microtubules or hollow fibers placed within the grooves. In general, a local anesthetic agent may be deployed at the proximal well of the tube 2 and transfer down the length of the tube 2. As the anesthetic agent moves through the microtubules or within the grooves present along the length of the tracheal tube, it may be released into the lumen of patient's trachea.

In some embodiments, the local anesthetic agent may be sealed within the proximal well 22 during manufacture and may be released during use by, for example, breaking a seal within the proximal well. In other embodiments, the proximal well may include a port such as a Luer or other fitting used to receive the local anesthetic agent from an external device, such as a syringe, infusion pump, pressurized container, or aerosolized container or apparatus. Through the use of such external device, an administrator may initiate, continue, cease, or resume the administration of the local anesthetic agent during intubation as needed using the port.

In some embodiments, the anesthetic agents may be delivered by nebulization. The anesthetic agents may be delivered as liquid nebulizers and dry nebulizers. In liquid nebulizers, the anesthetic agent in liquid form is nebulized into fine aerosol droplets and released. In dry nebulizers, solid and preferably powdered anesthetic preparations in very finely dispersed form are converted into a gaseous state and released as a dry mist. For example, the microtubules disclosed herein may introduce the anesthetic agent to the surrounding tissue of the intubated in a nebulized form. Thus, in some embodiments, the treacheal tube may include a compressor or pump for forcing air through a ventruri to a baffle and into the reservoir where the anesthetic agent can be nebulized and carried through the microtubules and delivered to the tissue surrounding the implanted tracheal tube. In other embodiments, the microtubules may include holes that are capable of acting as nozzles creating a soft mist when pressurized anesthetic agent is forced through the microtubules. In either embodiment, nebulization can be carried out continuously by introducing pressurized air or anesthetic agent to the microtubules or intermittently. Without wishing to be bound by theory, nebulization of the anesthetic agent may allow for more complete dispersion of the anesthetic agent with less irritation to the patient.

Formulations for nebulization may include anesthetics and analgesics described above, along with other pharmaceutically acceptable formulation excipients, including but not limited to coatings, stabilizers, emulsifiers, surfactants, and the like. Although not required, incorporation of a compatible surfactant can improve the stability of the respiratory dispersions, increase pulmonary deposition and facilitate the preparation of the suspension. Moreover, by altering the components, the density of the particle may be adjusted to approximate the density of the surrounding medium and further stabilize the dispersion. Any suitable surface active agent (surfactant) may be used in the context of the present invention, provided that the surfactant is preferably physiologically acceptable. Physiologically acceptable surfactants are generally known in the art and include various detergents and phospholipids, as discussed in more detail below. In accordance with one aspect, it is preferred that the surfactant is a phospholipid including, but not limited to, an extract of a natural surfactant such as any number of known pulmonary surfactants, including bovine- and calf-lung surfactant extracts, an egg phospholipid, a vegetable oil phospholipid such as a soybean phospholipid, or phosphatidylcholine. Preferably, in accordance with aspects of the present disclosure, the surfactant suitable for use with the therapeutic compositions of the present disclosure is an extract of a natural surfactant, an egg phospholipid, or combinations thereof. More preferably, the compositions may any one or more of a number of biocompatible materials as surfactants, such as surfactants comprising phospholipids.

The tracheal tubes described above can be used for delivery various types of materials and in particular embodiments, the tracheal tubes may be used for the delivery of one or more local anesthetic agents. The particular local anesthetic agent used can vary among embodiments, and the local anesthetic agents can include, but are not limited to, lidocaine, dibucaine, prilocaine, novocaine, other local anesthetic agents of this class, or combinations or mixtures thereof. Other non-limiting examples of local anesthetics include bupivacaine, etidocaine, tetracaine, lidocaine, xylocaine and salts, Versed, Ativan, propofol, flumazenil, thiopentone, Retairiine, remifentanyl, midazolam, pentothal, propofol, evipal procaine, nitrous oxide, methoxy flurane, sevoflurane, isoflurane, desflurane, ethylene, cyclopropane, ether chloroform, halothane, and the like.

The tracheal tubes of various embodiments described above may further include additional features as known in the art. For example, the tracheal tubes may include an adaptor attached to its proximal end that is configured to form a seal between the tracheal tube and a ventilator tube or air bag. In some embodiments, the adaptor may include a flange that that can be attached to the patient with an adhesive or tape after intubation to hold the tracheal tube in place. In other embodiments, the adaptor may include a strap or band that can be placed around the patients head after intubation to hold the tracheal tube in place. In still other embodiments, the tracheal tube may include one or more cuffs, as disclosed above, on a distal portion of the tube that can be expanded to hold the tracheal tube in place during use and avoid back flow of air up the trachea and away from the lungs.

Other embodiments are directed to methods for making a tracheal tube. Such methods may include machining a plurality of grooves in an outer surface of a tracheal tube and fitting microtubules or hollow fibers into the grooves. The method may further include sealing a proximal end of the microtubules or hollow fibers into a proximal well and sealing the distal ends of the microtubules or hollow fibers. In other embodiments, after machining a plurality of grooves on an outer surface of a tracheal tube, the method may include the step of attaching a semi-permeable membrane to the exterior surface of the tracheal tube over the grooves to form canals for flow of fluid behind the semi-permeable membranes. Such methods may further include the steps of sealing the proximal end of the grooves in a proximal well. Any of the methods described above may further include the steps attaching one or more adapters, flanges, cuffs, or other additional components to the tracheal tube.

Further embodiments are directed to methods for intubating a patient. Such embodiments, may include the step of intubating the patient with a tracheal tube having enclosed grooves as described above, and administering a pharmaceutical agent to the patient through the enclosed grooves. In some embodiments, the pharmaceutical agent may be an such as, for example, lidocaine, dibucaine, prilocaine, novocaine, other local anesthetic agents of this class, or combinations or mixtures thereof. In particular embodiments, the method may further include sedating the patient before, during, or after intubating. However, in certain embodiments, sedating the patient is not required, and intubating can occur without sedating the patient. Such embodiments, may include any other steps commonly performed during or after intubation such as, for example, attaching an adaptor onto the tracheal tube, attaching a ventilator line or ventilator bag to the tracheal tube, attaching the tracheal tube to the patient, and the like and combinations thereof.

The tracheal tubes as provided herein may be disposable or reusable, and capable of providing differential mechanical ventilation to either or both lungs, and capable of supporting all other functions of standard endotracheal tubes (e.g. sealing, positive pressure generation, suctioning, irrigation, drug instillation, etc). The tracheal tubes can be used in conjunction with all acceptable auxiliary airway devices such as (e.g. heat and humidity conservers, mechanical ventilators, humidifiers, closed suction systems, scavengers, capnometers, oxygen analyzers, mass spectrometers, PEEP/CPAP devices, etc). Furthermore, although the embodiments of the present disclosure illustrated and described herein are discussed in the context of tracheal tubes such as endotracheal tubes, it should be noted that presently contemplated embodiments may include a pressure distribution lumen used in conjunction with other types of airway devices. For example, the disclosed embodiments may be used as a single-lumen tube, tracheostomy tube, a double-lumen tube (e.g., a Broncho-Cath™ tube), a specialty tube, or any other airway device with a main ventilation lumen. The endotracheal tubes may be adapted for use with any suitable patient. Patients may include animals or humans of any suitable size, including infants and neonates.

1. A tracheal tube comprising:
a tube having longitudinally extending grooves on an exterior surface of the tube, each of the longitudinally extending grooves terminating in a closed end;
a semipermeable membrane enclosing the longitudinally extending grooves.

2. The tracheal tube of claim 1, wherein the semipermeable membrane has a molecular weight cut-off of about 100 Da to about 1500 Da.

3. The tracheal tube of claim 1, further comprising a proximal well enclosing a proximal end of each of the longitudinally extending grooves.

4. The tracheal tube of claim 3, wherein the proximal well contains a medicament.

5. The tracheal tube of claim 2, wherein the semipermeable membrane has a sieving coefficient of about 0.6 to about 0.7 for the medicament.

6. The tracheal tube of claim 3, further comprising a port positioned to allow access to the proximal well.

7. The tracheal tube of claim 1, further comprising a cuff.

8. A tracheal tube comprising:
a tube having longitudinally extending grooves on an exterior surface of the tube, each of the longitudinally extending grooves terminating in a closed end;
mircotubules having a plurality of holes fitted within each of the longitudinally extending grooves.

9. The tracheal tube of claim 8, further comprising a proximal well enclosing a proximal end of each of the microtubules.

10. The tracheal tube of claim 9, wherein the proximal well contains a medicament.

11. The tracheal tube of claim 9, further comprising a port positioned to allow access to the proximal well.

12. The tracheal tube of claim 8, further comprising a cuff.

13. The tracheal tube of claim 8, wherein the holes are sized to cause nebulization of a medicament in the microtubules.
